(19) United States
(12) Patent Application Publication
(10) Pub. No.: US 2012/0221035 A1
(43) Pub. Date: Aug. 30, 2012

(54) SURGICAL CUTTING ACCESSORY WITH FLEXIBLE TUBE

(76) Inventor: Stephen J. Harvey, Mallow (IE)

(21) Appl. No.: 13/408,385

(22) Filed: Feb. 29, 2012

Related U.S. Application Data
(63) Continuation of application No. PCT/EP2010/005318, filed on Aug. 30, 2010.
(60) Provisional application No. 61/275,518, filed on Aug. 31, 2009.

(51) Int. Cl. A61B 17/32

(52) U.S. Cl. 606/170

(57) ABSTRACT

A surgical cutting accessory for attachment to a powered surgical handpiece having a cutting element for cooperation with a drive member of the handpiece, a distal end defining a cutting element, and an elongate tube disposed between the proximal and distal ends. The tube of the cutting element has a flexible portion defined by a continuous cut disposed in the tube and extending in a helical manner generally axially, and a tubular liner is disposed within the tube axially adjacent the flexible portion. The accessory additionally having an outer tube housing in which the cutting element is disposed, the outer tube having a bent portion.

This Application is a continuation of International Application No. PCT/EP2010/005318 filed 30 Aug. 2010 which designated the United States. The '318 PCT Application is a non-provisional application based on and that claims priority from U.S. Pat. App. No. 61/275,518 filed 31 Aug. 2009. The contents of the above-identified application are explicitly incorporated herein by reference.

### FIELD OF THE INVENTION

This invention relates generally to a surgical cutting accessory that is used to cut or manipulate tissue and, more particularly, to a surgical cutting accessory having a flexible tube.

### BACKGROUND OF THE INVENTION

The goal of many surgical procedures is to remove and/or shape body tissue at the site at which the procedure is performed. Such procedures may involve surgery in the nasal and sinus cavities or in the throat, wherein this type of selective removal or shaping of tissue is performed. For example, sinus surgery often involves the removal of diseased membranes, bone partitions and/or malformed portions of sinus tissue, sometimes referred to as the sinus layer, and bony material entrained in this layer. Further, orthopedic surgery involves the shaping of bones and soft tissue that form the joints of the skeletal system, and arthroscopic surgery involves shaping or removal of hard and soft bodily tissues within the knee, shoulder and other joints.

The ability to perform the above-mentioned types of surgical procedures is enhanced by the development of powered surgical tools especially designed to perform such procedures. For example, the Assignee hereof manufactures a line of surgical tools under the trademarks HUMMER and ESSx that are especially designed to perform nasal, sinus and throat surgery. This line of tools includes a handpiece with an electrically driven motor. Different cutting accessories are designed for connection to the handpiece and are driven by the motor. Each cutting accessory typically has a hollow rotating or reciprocating inner tube that is housed in a fixed and hollow outer housing. In many surgical procedures, irrigating fluid is introduced into the surgical site and serves as a transport media for removing tissue and debris from the surgical site. In order to remove the irrigating fluid and the material contained therein, the above handpiece and the various accessories which are usable therewith together define a suction conduit. A suction pump is connected to the handpiece to provide the suction force needed for drawing the fluid and material away from the surgical site. In order to control the suction flow through the accessory and the handpiece, the handpiece is provided with a manually operated valve which is manipulated by the surgeon to control suction of material away from the surgical site. In this line of tools, irrigating solution is supplied to the distal end of the cutting accessory through an annular space between the inner tube and the outer housing of the cutting accessory. This fluid is then drawn away from the surgical site by suction which is applied through the inner tube of the accessory.

Another surgical tool sold by the Assignee hereof under the trademark FORMULA is designed for performing arthroscopic procedures on the knee or other bodily joints. This tool also incorporates an on-board motor and a coupling assembly for releasably holding a cutting accessory driven by the motor, and provides suction through the handpiece and the attached accessory. Irrigating fluid is supplied to the surgical site via a separate tool, for example, to distend the joint.

A common type of cutting accessory utilized with the above tools, often termed a “shaver”, includes a hub which defines the proximal end of the accessory and is appropriately configured to cooperate with the handpiece to lock the accessory thereto, an elongated and fixed tubular housing element having a proximal end fixed to the hub, and an elongated cutting element including a rotating drive tube disposed within the housing element. When the accessory is attached to the handpiece, the handpiece motor couples to the drive tube of the accessory and moves same relative to the outer housing element. The handpiece motor is selectively actuable to drive the accessory drive tube so as to cause a desired cutting action at the distal end of the accessory. The handpiece is associated with a control unit which controls the functioning thereof, and is actuated by the user via appropriate buttons provided on the handpiece itself, or alternatively directly at the control unit.

In such a shaver-type cutting accessory as described above, the outer housing element defines a window or opening at the distal end, which window is defined by an edge of the wall of the outer housing element. The cutting element drive tube at the distal end thereof also defines a window defined by an edge of the wall of the drive tube, and when the drive tube is disposed within the housing element, the drive tube window is positioned adjacent the window of the housing element. As the drive tube is moved relative to the housing element by the handpiece motor, the cutting edge of the drive tube window and the opposed and facing cutting edge of the housing element window cause a cutting action which effectively severs tissue located within the housing element window and between the opposed cutting edges of the housing element and drive tube. The configurations of these opposed edges allow for removal of particular tissue types, and a variety of different blade geometries are available to specifically address the type of cutting the accessory is to carry out. Other types of cutting accessories are usable with the above tools, such as a bur, which can include a suction opening at its distal end adjacent the working tip of the bur to permit suction to be applied to the surgical site.

The cutting accessories usable with the above handpieces may be straight or angled, depending upon the type of procedure to be performed. In many surgical procedures, it is desirable to have an angled or curved accessory so as to allow access to surgical sites which are generally not accessible with straight cutting accessories. For example, curved or angled accessories are often necessary in sinus or throat surgeries, due to the natural curvatures of these areas of the body. Further, in arthroscopic knee surgery, it is often desirable to use curved cutting instruments. Thus, cutting accessories with curved or bendable tubes are known. One such accessory utilizes an inner rotary tube having a spirally-cut portion to impart flexibility and allow the inner tube to conform to the curved shape of the outer fixed housing. A wrap formed from a helically or spirally wound strip of material is superimposed over the spirally-cut portion of the inner tube, and is wrapped around the tube in a direction opposite to the turn or slant of the helical cut. This arrangement defines a flexible region in the inner tube which transmits torque from a motorized handpiece, while allowing the inner tube to conform to the bent shape of the outer housing. Suction can be applied to the surgical site through the hollow interior of the inner tube.

While the known flexible surgical accessories have proven useful for their intended purpose, such accessories, by providing a wrap over the helically-cut tube, can be complicated to manufacture and necessarily result in a larger-diameter inner tube and a larger-diameter outer fixed housing which must accommodate the inner tube. In this regard, it is desirable to minimize the outer diameter of the accessory tube so as to minimize trauma to the patient.

In order to minimize or obviate the disadvantages of known surgical cutting accessories, the surgical cutting accessory according to one embodiment of the invention incorporates an outer fixed housing element in which an inner cutting element is movably disposed. The inner cutting element has an elongate and tubular tube in which a continuous helical cut is provided to allow the inner cutting element to conform to an angled or curved configuration of the outer housing element. In order to provide adequate suction through the inner tube, a tubular liner is located within the inner tube along the flexible portion thereof, which liner effectively seals the continuous opening along the inner tube which is created when the inner tube is cut. Further, in surgical tools which provide irrigation fluid in the annular space between the outer diameter of the inner tube and the inner diameter of the outer housing element, the liner maintains the irrigation and suction passages wholly separate from one another and prevents leaking of fluid between these passages.

The invention may also be incorporated into surgical drills, which include a handpiece with an on-board motor, and various complementary attachments. The primary difference between surgical “shavers” and surgical “drills” is that a surgical drill is typically able to operate at higher speeds than a shaver-type handpiece.

The attachments mentioned above serve as the head of the handpiece to which a cutting accessory is attached. Some attachments are provided with linkages which transfer the motive power from the handpiece motor to the cutting accessory, for example, to extend the length of the handpiece. Other attachments function as bent-angle units, which attachments orient the associated cutting accessory at an angle away from the axis of the handpiece so as to provide the surgeon with an alternative view of the surgical site. Further, some attachments translate the rotary motion of the handpiece motor into either a reciprocating or oscillatory motion. Still other attachments function to provide physical support for a cutting accessory which rotates along with the handpiece motor, to prevent the tube of the cutting accessory from bending when exposed to significant loads during surgery.

Some attachments may also include what is commonly called a “nose” which is bent at a selected angle and supports an accessory embodied by a flexible wire which extends through the nose and defines a cutting head at a distal end of the wire. Thus, the inner cutting element according to the invention provided with a continuous helical cut for flexibility may be provided as a cutting accessory within a bent nose of a surgical drill so as to conform thereto. The tubular liner can be located within the inner cutting element to provide structural integrity thereto. Additionally, the attachment may be appropriately ported to permit suction and/or irrigation at the surgical site, in which case the liner located within the inner cutting element serves to seal a suction passage defined within the cutting accessory, and, if irrigation is provided in the annular passage between the nose tube and the inner cutting element, the liner serves to maintain the suction and irrigation passages separate from one another.

### BRIEF DESCRIPTION OF THE DRAWINGS

FIG. 1 is a fragmentary side view of a surgical cutting accessory according to the invention;

FIG. 2 is an exploded view of the outer housing element and the inner cutting element of the surgical cutting accessory of FIG. 1, prior to assembly and bending of the outer housing element;

FIG. 3 is an exploded view of the tube and liner of the inner cutting element of the surgical cutting accessory of FIG. 1;

FIG. 4 is an enlarged and fragmentary perspective view of the distal end portion of the inner cutting element of the surgical cutting accessory of FIG. 1;

FIG. 5 is an enlarged and fragmentary perspective view of the flexible portion of the inner cutting element of the surgical cutting accessory of FIG. 1;

FIG. 6 is an enlarged and fragmentary view of the flexible portion of the tube of the inner cutting element, with the material of the tube shown in a flattened condition to illustrate the pattern of the continuous cut formed therein;

FIG. 6A is an enlarged and fragmentary view of the helically cut geometry of the flexible portion of the inner cutting tube, with the material of the tube shown in a flattened condition to illustrate the pattern of the continuous cut formed therein;

FIG. 6B is a further enlarged and fragmentary view of the helically cut geometry of the flexible portion of the inner cutting tube, with the material of the tube shown in a flattened condition to illustrate the pattern of the continuous cut formed therein;

FIG. 7 is an enlarged, fragmentary longitudinal cross-sectional view of the distal end of the inner cutting element of the surgical cutting accessory of FIG. 1.

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the arrangement and designated parts thereof. The words “forwardly” and “distally” will refer to the direction toward the end of the arrangement which is closest to the patient, and the words “rearwardly” and “proximally” will refer to the direction toward the end of the arrangement which is furthest from the patient. Said terminology will include the words specifically mentioned, derivatives thereof, and words of similar import.

### DETAILED DESCRIPTION

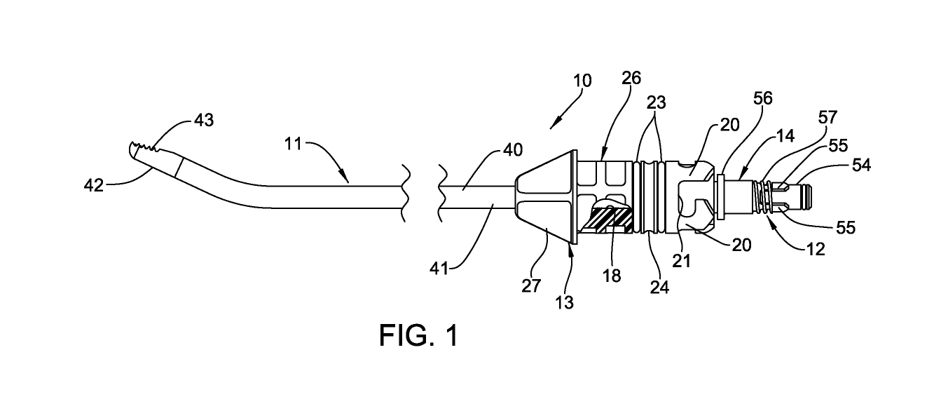

FIGS. 1-3 illustrate a surgical cutting accessory 10 according to the invention. Cutting accessory 10, in the illustrated embodiment, is a surgical shaver, and includes an outer tubular housing element 11 and a tubular cutting element 12 disposed within housing element 11. Housing element 11 includes a hub 13 which defines the proximal end of housing element 11 and is configured to releasably hold the cutting accessory 10 to a surgical handpiece (not shown), with which the cutting accessory 10 is used. Cutting element 12 also includes a hub 14 which defines the proximal end of element 12 and is configured for driving engagement with a drive member of the surgical handpiece. Examples of such handpieces are sold by the Assignee hereof under the following trademarks: Hummer 4; ESSx Microdebrider; and TPS.

The hub 13 of housing element 11 is described in U.S. Pat. No. 6,958,071, which is owned by the Assignee hereof and which is hereby incorporated by reference in its entirety herein. Hub 13 will accordingly be only briefly described here. Hub 13 is a generally tubular member which defines a longitudinally-extending bore 18 therein which extends completely axially through hub 13 and opens both proximally and distally. Hub 13 is formed to define a plurality of circumferentially-spaced and generally L-shaped teeth 20 at its proximal end. Teeth 20 are shaped so as to define respective generally L-shaped lock slots 21 between adjacent pairs of teeth 20. Distally from teeth 20, hub 13 defines therein a pair of axially-spaced and circumferentially extending grooves in which respective O-rings 23 are seated, and a shallow, concave and circumferentially extending groove 24 disposed axially between the grooves in which O-rings 23 are disposed. A bore 25 extends radially from the base of groove 24 to the inner axially extending bore 18 of hub 13 for communication therewith. Hub 13 is formed with a webbed-configuration 26 axially adjacent the most distal of the O-rings 23 and generally triangularly-shaped webs 27 which define the distal end of hub 13. Webbed configuration 26 and distal webs 27 provide structural strength to the hub 13. Again, it should be understood that the specific geometries of hubs 13 and 14 are not material to the present invention.

Outer housing element 11 additionally includes an outer tube 40 having a proximal end 41 fixed within a distal end of housing bore 18 of hub 13, and a distal end 42 which is at least partially closed in the axial direction and which defines therein a window 43 which opens generally sidewardly or transversely to a longitudinal axis of outer tube 40. Outer tube 40 is hollow along its length so as to define a conduit 44 therein with which window 43 communicates. Window 43 defines a cutting window, and in the illustrated embodiment, window 43 is toothed on opposite longitudinal sides thereof.

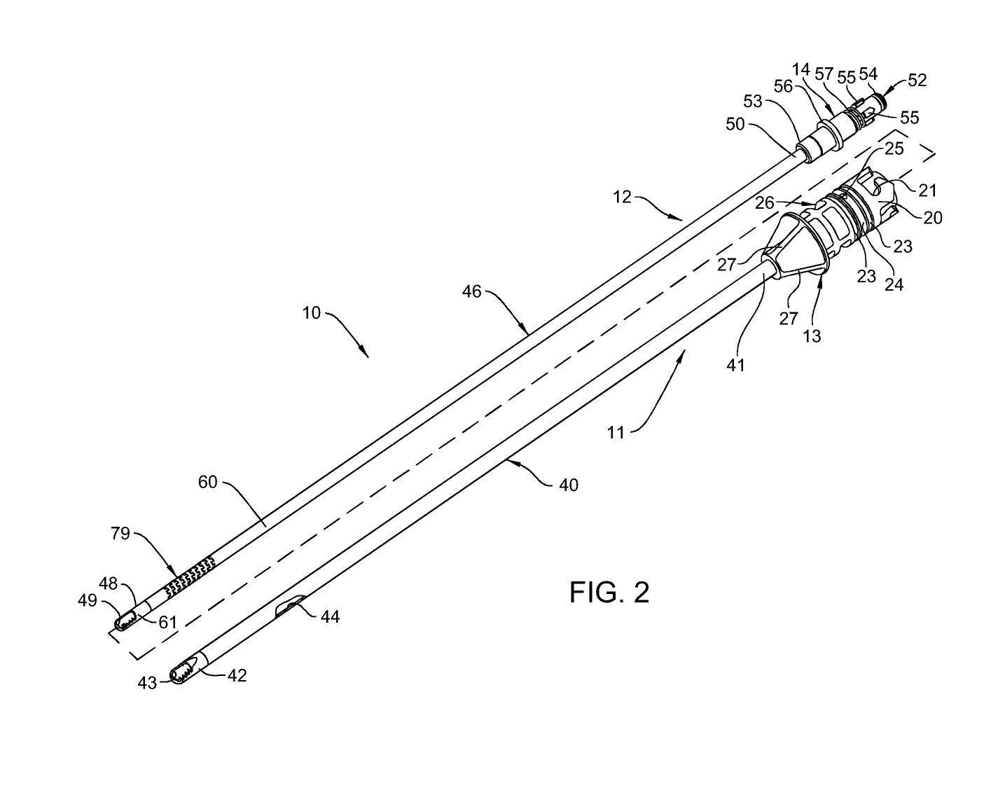

Turning now to cutting element 12, same includes an inner tube 46 which defines a conduit 47 along the length thereof. The inner tube 46 is formed from 304 stainless steel. The inner tube 46 disposed within the outer tube 40. If the outer tube 40 has a diameter of 4.1 mm, the inner tube 46 will have an outer diameter of 3.2 mm. This inner tube 46 will have a wall thickness of approximately 0.25 mm. Alternatively, if the outer tube 40 has an outer diameter of 3.2 mm, then the inner tube 46 will have an outer diameter of 2.7 mm. This alternative inner tube 46 will have a wall thickness of approximately 0.22 mm. Inner tube 46 has a distal end 48 which is closed in the axial direction. Distal end 48 defines therein a sidewardly-opening window 49 which communicates with conduit 47 of inner tube 46. Window 49, in the illustrated embodiment, is toothed on opposite longitudinal sides thereof and defines a cutting window. As shown in FIG. 2, inner tube 46 additionally includes a proximal end 50 which is fixed to hub 14. Hub 14 is generally cylindrical and defines therein a bore 52 which extends axially completely through hub 14 and opens both proximally and distally. Hub 14 has a distal end portion 53 in which the proximal end 50 of inner tube 46 is fixed, and a proximal end portion 54 joined to distal end portion 53 and on which a plurality of longitudinally extending and circumferentially spaced-apart teeth 55 are disposed. An annular collar or flange 56 is disposed axially between distal and proximal end portions 53 and 54. A spring 57 is disposed about proximal end portion 54 distally from teeth 55.

In the illustrated embodiment, inner tube 46 is a two-piece component including a proximal section 60 and a tip 61. Tip 61 defines window 49 and closed distal end 48. Tip 61 may initially be formed from a tube which is closed at one axial end, wherein window 49 is cut into the side thereof, and at the other axial end is fixed, for example by welding, to a terminal tubular distal end 62 of proximal section 60. Outer tube 40 of outer housing element 11 may be constructed in a similar, i.e. two-piece, manner.

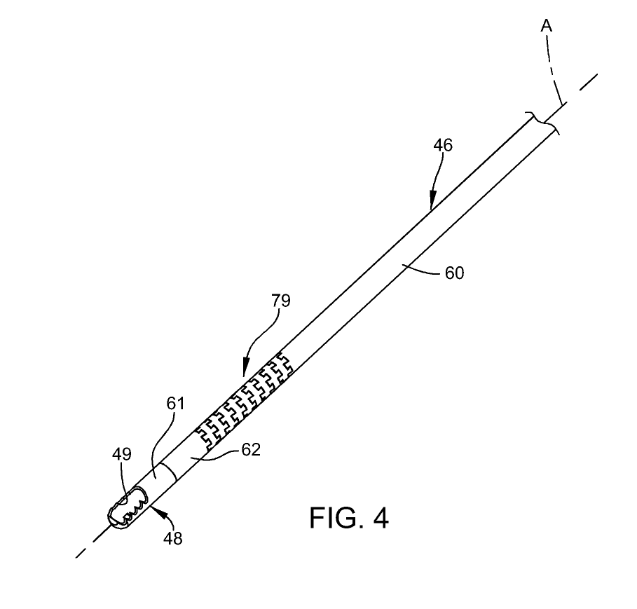

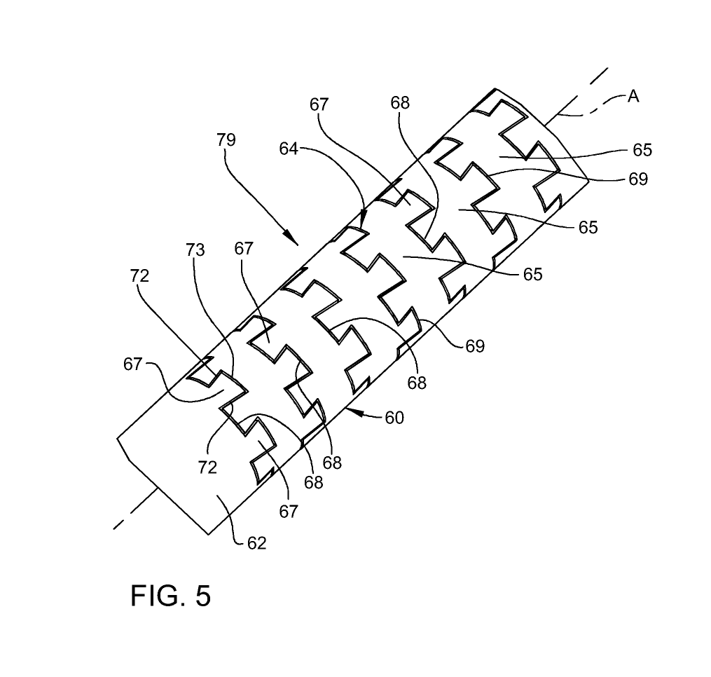

Referring to FIGS. 4-6, proximal section 60 of inner tube 46, adjacent the distal end 62, defines therein a single continuous cut 64 which is formed in the shape of a helix about the circumference of section 60 and radially completely through the wall of section 60 of inner tube 46. The cut 64 extends in a path about a central longitudinal axis A of inner tube 46, such that opposite proximal and distal terminal ends of the cut 64 are axially spaced and do not meet one another. The formation of cut 64 in inner tube 46 effectively creates a plurality of generally helical tube segments or sections 65, each of which extends about the full circumference of inner tube 46 and adjoins the next adjacent section 65. The helix angle of cut 64 results in the segments 65 being disposed at an angle relative to axis A of inner tube 46. In a preferred embodiment of the invention, this angle is 80 degrees.

Generally, the width of the cut 64 is between 0.01 mm to 0.04 mm. In some preferred versions of the invention, the width is between 0.02 mm and 0.03 mm. In the illustrated embodiment, the helical cut 64 is formed from a number of smaller cuts 92-95. Cuts 92-95 form a plurality of axially adjacent interlocking dovetails 90 and 91 in a flexible portion 79 of the inner tube 46. Each dovetail 90 and 91 includes, respectively, a top cut 93 or a bottom cut 92. Two opposed side cuts 94 and 95 extend to the bottom cut 92 or top cut 93. Each dovetail 90 and 91 is perpendicular to the longitudinal axis A of the inner tube 46. In other words, the bottom and top cuts 92 and 93, respectively, extend arcuately along the flexible portion 79 and perpendicular to axis A. Side cuts 94 and 95, which also define the dovetails 90 and 91, extend away, at an angle, from the bottom and top cuts 92 and 93. Side cut 94 is a long cut. Side cut 95 is a short cut. Generally, the long cut 94 has a length that is 1.3 to 2.3 times the length of short cut 95. In some versions of the invention, long cut 94 has a length between 1.4 and 1.6 times the length of short cut 95. It should be appreciated that the difference in length between the long and short cuts, 94 and 95, respectively, is what determines the angle of the helical cut 64 relative to the longitudinal axis of inner tube 46.

Angle α defines the angle formed by the bottom cut 92 or top cut 93 with the adjacent side cuts 94 and 95. Angle α is typically between 75 degrees and 85 degrees. Angle α is typically the same for each interface between the bottom and top cuts and the adjacent side cuts.

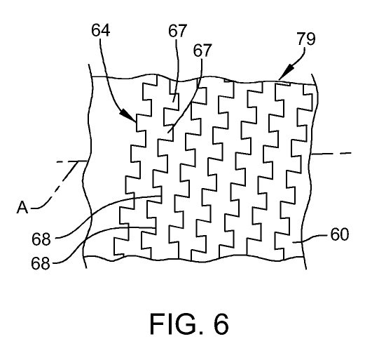

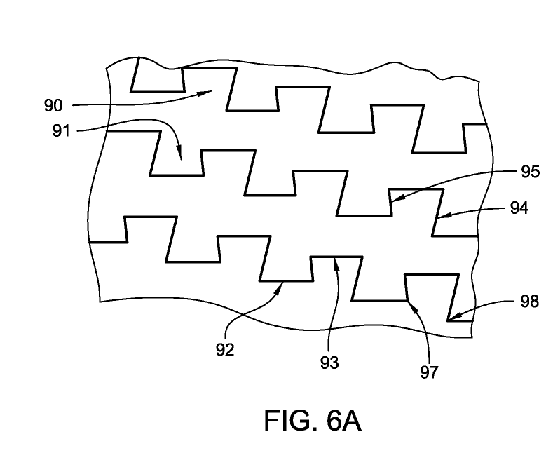

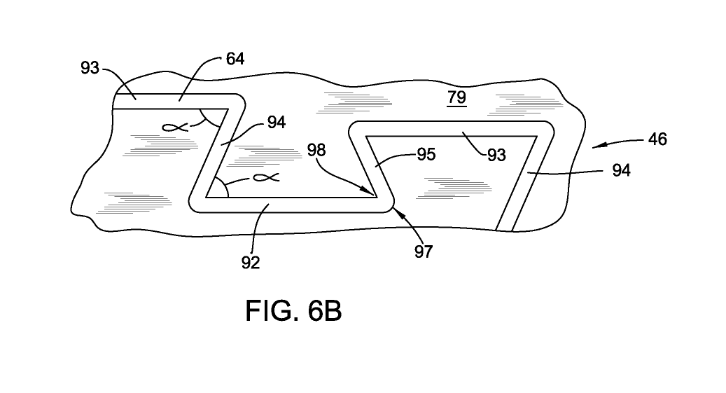

As best shown in FIG. 6B, each dovetail 90 and 91 is defined by a rounded corner 97 and an angled corner 98. Angled corner 98 is the corner around the point of the dovetail where the side cut and bottom (or top) cut meet to form the actual corner of the dovetail 90 and 91. Rounded corner 97 is the edge of the tube section opposite the cuts that surround the angled corner 98. The edges of this section of the tube come together along an interface that is more rounded than of the edges that define the angled corner 98. Rounded corner 97 can have a radius of curvature from 0.005 mm to 0.03 mm.

When a dovetail 90 and 91 is subjected to side loading, a significant amount of the force is imposed on the portion of the dovetail 90 and 91 that emerges from the rest of the inner tube 46. Instead of this force being focused on a point, it is distributed over rounded corner 97. This reduces the likelihood of the shear force causing a stress fracture in the dovetail 90 and 91. Such fracturing, if allowed to occur, could appreciable adversely affect the ability of the flexible portion 79 to transfer torque.

Angled corners 98 have the same value as previously identified angle α. It can also be appreciated that the angled corner 98 and rounded corner 97 are angled and rounded, respectively, due to manufacturing reasons. In alternative embodiments of the invention, each dovetail 90 and 91 can be defined by two rounded corners.

When inner tube 46 rotates, each dovetail 90 and 91 presses against the adjacent dovetail that leads in the direction of rotation of inner tube 46. This dovetail-against-dovetail abutment is what causes the tube flexible portion 79 to transfer appreciable amounts of torque from hub 13 to tip 61. Owing to the orientation of the dovetails relative to the shaft longitudinal axis, this dovetail-against-dovetail abutment is the same regardless of the direction of tube rotation. This is especially important for versions of this invention constructed as a shaver. This is because a shaver is often operated in an oscillating mode. The inner tube 46 is typically rotated 360 degrees to 2200 degrees in one direction and then rotated back through the same number of degrees in the reverse direction. When the shaver is operated so that, in one cycle it rotates 1800 degrees (five complete 360 degree rotations) in one direction followed by 1800 degrees of rotation in the opposite direction, the inner tube 46 can undergo 200 or more cycles per minute. The interlocking of the dovetails 90 and 91 under these operating conditions again is what facilitates the torque transfer to tip 61. Moreover, as a consequence of this particular abutment of the dovetails 90 and 91, the dovetails are relatively tightly interlocked. This interlocking of the dovetails 90 and 91 reduces the likelihood of the rotation of the tube resulting in the disengagement of the dovetails.

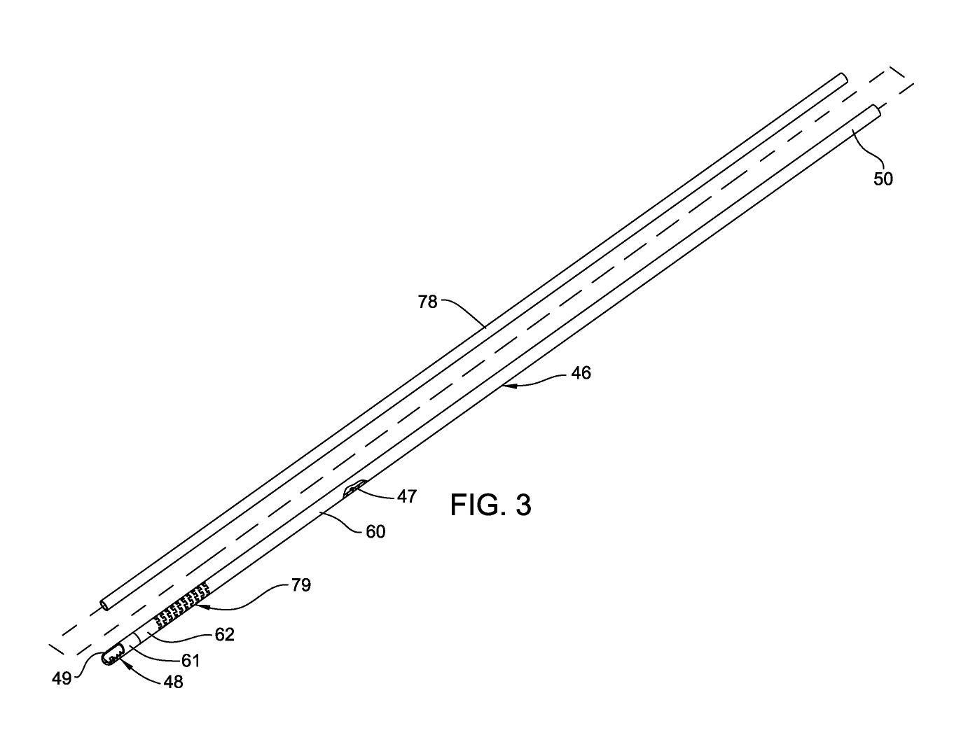

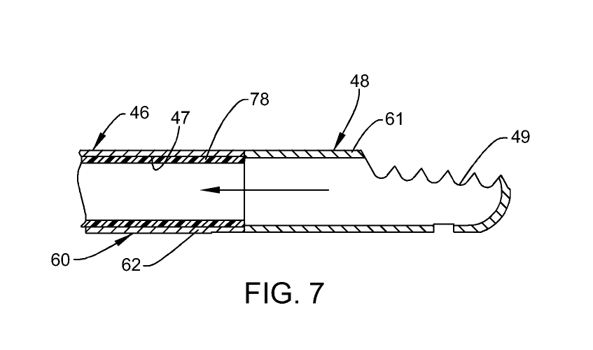

Referring to FIGS. 3 and 7, cutting element 12 additionally includes a generally tubular liner 78 located within conduit 47 thereof. Generally, the tubular liner 78 has a thickness ranging from 0.015 mm to 0.06 mm. In some preferred versions of the invention, the tubular liner 78 has a thickness ranging from 0.02 mm to 0.05 mm. Specifically, liner 78 is inserted into the proximal end 50 of inner tube 46, for example, using a mandrel, so that liner 78 extends at least axially along the flexible portion 79 of inner tube 46 as defined by cut 64. In the illustrated embodiment, the liner 78 extends substantially along the entire longitudinal extent of inner tube 46. It will be appreciated that liner 78 may only extend along flexible portion 79 of inner tube 46. Adhesive may then be used to securely fasten liner 78 within inner tube 46, for example, at opposite axial ends of liner 78 between the outer surface of liner 78 and the inner surface of inner tube 46 which defines conduit 47. A biocompatible adhesive is used to connect the liner 78 with the inner tube 46.

Cutting element 12 is assembled to outer housing element 11 by inserting the distal end 48 of element 12 into the open proximal end of hub bore 18 and into conduit 44 of outer tube 40, and advancing the cutting element 12 in the distal direction relative to housing element 11 until the collar 56 of hub 14 abuts or is axially adjacent the terminal proximal end of hub 13. With the cutting element 12 in this position inside outer housing element 11, the cutting windows 49 and 43 of the respective tubes 46 and 40 are located axially adjacent one another. Once the cutting element 12 is assembled to the outer housing element 11, the outer tube 40 can be bent to a desired angular orientation such as that shown in FIG. 1, at the area where outer tube 40 axially overlaps the flexible portion 79 of inner tube 46, using an appropriate tool.

Similarly, there is no requirement the flexible portion 79 of the inner tube 46 always be located adjacent the distal end 42 of outer tube 40. In some versions of the invention, the bend in the outer tube 40 is located in the middle of the outer tube 40. The flexible portion 79 of the inner tube 46 would likewise be located in the middle of the inner tube 46.

The attachment of the cutting accessory 10 to the surgical handpiece is described in the '071 patent referenced above, and will accordingly be only briefly described here. With the cutting element 12 installed within outer housing element 11 as discussed above, the hubs 13 and 14 are inserted into the open distal end of the handpiece which causes seating of pins provided on the handpiece in the slots 21 of hub 13, which effectively holds the cutting accessory 10 to the handpiece. The insertion of the accessory 10 into the handpiece also causes coupling of the hub 14 of cutting element 12 to the appropriate gear train head or drive member of handpiece, wherein the teeth 55 of hub 14 engage with teeth provided on the gear train head. Thus, when the handpiece is actuated, the handpiece motor causes rotation of the inner tube 46 of cutting element 12 relative to and within outer tube 40 of outer housing element 11. This relative motion between tubes 40 and 46 causes the cutting window 49 of cutting element 12 to rotate past window 43 of outer tube 40, so that the toothed cutting edges of windows 43 and 49 effectively cut tissue located within or adjacent window 43. In this regard, when the accessory 10 is installed on the handpiece, the spring 57 provided on hub 14 extends between the gear train head of the handpiece and the hub 14, and urges hub 14 and associated inner tube 46 forward so that the distal end 48 of inner tube 46 is in bearing contact with the distal end 42 of outer tube 40.

During a surgical procedure, suction may be drawn through the handpiece, through the cutting element inner tube 46, and through window 43 of outer tube 40 of outer housing element 11. Thus, surgical debris can be suctioned away from the surgical site, and this suction can be controlled by manipulating a valve provided on the handpiece. The liner 78 serves to seal the continuous gap 69 created by the cut 64 provided in inner tube 46, and thus suction is drawn through the interior of the liner 78 rearwardly or proximally into the handpiece as shown by the arrow in FIG. 7. The liner 78 is constructed of a biocompatible plastic, such as PTFE, FEP or PET. Other materials may be utilized in accordance with the invention, provided that such materials adequately seal the interior of the flexible portion 79 of inner tube 46.

Irrigating fluid may also be supplied to the surgical site via handpiece and an associated pump as described in U.S. Pat. No. 6,958,071. In this regard, when irrigating fluid is forced through the handpiece by the pump, such fluid is forced into bore 25 and annular groove 24 of hub 13, and then into the annular space defined between the outer surface of inner tube 46 and the inner surface of outer tube 40 which defines conduit 44 and out window 43 to the surgical site. The O-rings 23 provided on hub 13 prevent flow of the irrigating fluid into the handpiece bore. It will be appreciated that irrigation fluid may alternatively be provided via a separate irrigation tool, instead of via the handpiece as described above.

Windows 43 and 49 of the respective tubes 40 and 46 of cutting accessory 10 are illustrated herein with a toothed configuration, which may be utilized when an aggressive cutting action is required. It will be appreciated that other configurations may be provided, and the illustrated toothed configuration is presented only by way of example. For example, one cutting window 43 or 49 may be provided with a straight edge, and the other cutting window provided with a toothed edge, for a less-aggressive cut than that which is achieved with the illustrated toothed cutting windows. Further, the edges of the respective tubes 40 and 46 which define the respective windows 43 and 49 may both be provided with straight configurations, which may be used for a more precise cut.

Although particular preferred embodiments of the invention are disclosed in detail for illustrative purposes, it will be recognized that variations or modifications of the disclosed apparatus, including the rearrangement of parts, lie within the scope of the present invention.

For example, in some versions of the invention the angle a that defines the corners of the dovetails may vary. Likewise this invention is not limited to a shaver-type cutting accessory. The distal end tip attached to the flexible inner tube may not always be a window that is surrounded by sharp edges. In some versions of the invention, a bur head may be attached to this tip. The cutting edge of the bur functions as the cutting features of the tip. Below the bur head, the tip is formed with a window into the lumen that extends through the inner tube. Likewise in some versions of the invention, it may not be necessary to provide a liner around the section of the inner tube formed with the cut. Similarly, when a linear is provided, it may not extend the full length of the inner tube. Instead, it may be desirable to have the liner extend only over the flexible portion of the inner tube; the portion in which the cut is defined.

Also, there is no requirement that the inner tube assembly be formed from two pieces. In some versions of the invention, the elongated torque transfer tube with the helical cut and the tip with the window defined by the teeth may be formed as a single unit. Similarly, in some versions of the invention, the helix forming dovetails may be formed so that the cuts sections that define the corners of the dovetails themselves, instead of coming to a point, be rounded.

What is claimed is:

1. A surgical cutting accessory, said accessory including:
an inner hub having features for engaging a handpiece drive tube so that the drive tube can rotate the inner hub;
an inner tube extending distally away from said inner hub, said inner tube having a distal end spaced from the inner hub and having a longitudinal axis, a lumen that extends through the inner tube, and a flexible portion, said flexible portion defined by a cut disposed in said inner tube that extends helically axially along said inner tube;
a tip attached to the distal end of the inner tube, the tip having cutting features and a window into the inner tube lumen;
an outer hub;
an outer tube having a proximal end, the outer tube being disposed over the inner tube, the outer tube extending distally forward from the outer hub over the inner tube, the outer tube being bent over the inner tube flexible portion and having a window that is open adjacent the tip window,
wherein the helical cut said inner tube is shaped to form a plurality of axially adjacent interlocking dovetails in the flexible portion of said inner tube, each dovetail being defined by a bottom cut or a top cut and two opposed side cuts that extend to the bottom cut or top cut, the bottom cuts and top cuts being perpendicular to the longitudinal axis of the inner tube, where a first one of the side cuts extends a first length away from the bottom or top cut, and a second one of the side cuts extends a second length away from the bottom or top cut, the second length of side cut being greater than the first length of side cut.

2. The surgical cutting accessory of claim 1, wherein said cutting accessory further includes a tubular liner, said liner disposed within said inner tube axially adjacent said flexible portion.

3. The surgical cutting accessory of claim 2, wherein said tubular liner has a thickness ranging from 0.015 mm to 0.06 mm.

4. The surgical cutting accessory of claim 1, wherein said cutting accessory further includes:
a tubular liner; and
said conduit of inner tube is a suction conduit in communication with a suction source, said liner defining a portion of said suction conduit and forming a seal along said flexible portion of said inner tube between said suction conduit and said outer tube.

5. The surgical cutting accessory of claim 1, wherein the cut extends completely radially through said flexible portion of said inner tube.

6. The surgical cutting accessory of claim 1, wherein the width of the cut is between 0.01 mm to 0.04 mm.

7. The surgical cutting accessory of claim 1, wherein said inner tube is used for cutting and is disposed for movement relative to said outer tube, said outer tube having a distal end which defines a sidewardly-opening window therein to expose a plurality of teeth of said inner tube.

8. The surgical cutting accessory of claim 1, wherein said cutting accessory further includes a tubular liner, said tubular liner is attached within inner tube at opposite axial ends of said liner between the outer surface of said liner and the inner surface of said inner tube defining a conduit.

9. The surgical cutting accessory of claim 1, wherein at least one side cut and said bottom cut or top cut meet to define a dovetail, the cuts define in the dovetail an angled corner and, in the section of the inner portion of cut opposite the angled corner, a rounded corner.

10. The surgical cutting accessory of claim 9, wherein the radius of curvature of said rounded corner ranges from 0.005 mm to 0.03 mm.

11. The surgical cutting accessory of claim 1, wherein each side cut of each interlocking dovetail is angled away from a complementary bottom cut or top cut.

12. The surgical cutting accessory of claim 11, wherein the angle formed by each side cut with each complementary bottom cut or top cut ranges from 75 degrees to 85 degrees.

13. The surgical cutting accessory of claim 1, wherein the ratio between a first side cut and a second side cut is constant throughout the helical pattern of said cut.
